(19) United States
(12) Patent Application Publication
(10) Pub. No.: US 2025/0102434 A1
(43) Pub. Date: Mar. 27, 2025

(54) LIGHT SOURCE APPARATUS, INSPECTION APPARATUS, EXPOSURE APPARATUS, LIGHT SOURCE CONTROL METHOD, INSPECTION METHOD, AND EXPOSURE METHOD

(71) Applicant: Lasertec Corporation, Yokohama (JP)

(72) Inventors: Mizuki KOBAYASHI, Yokohama (JP); Keitaro HAYASHIDA, Yokohama (JP); Hirokazu SEKI, Yokohama (JP)

(73) Assignee: Lasertec Corporation, Yokohama (JP)

(21) Appl. No.: 18/894,572

(22) Filed: Sep. 24, 2024

(30) Foreign Application Priority Data
Sep. 25, 2023 (JP) . 2023-160000

(51) Int. Cl. G01N 21/64; G01N 33/205

(52) U.S. Cl. CPC G01N 21/64; G01N 33/205; G01N 2201/0636

(57) ABSTRACT

A light source apparatus according to the present disclosure includes an input optical system including a first optical member configured to irradiate a target material with excitation light, an output optical system including a second optical member configured to extract light generated by irradiating the target material with the excitation light, a target holding unit configured to hold the target material, an acquiring unit configured to acquire a surface position of the target material, a driving unit configured to cause a position of a focusing point of at least one of the first optical member and the second optical member to vary, and a control unit configured to drive the driving unit based on the surface position.

### INCORPORATION BY REFERENCE

This application is based upon and claims the benefit of priority from Japanese patent application No. 2023-160000, filed on Sep. 25, 2023, the disclosure of which is incorporated herein in its entirety by reference.

### BACKGROUND

The present disclosure relates to a light source apparatus, an inspection apparatus, an exposure apparatus, a light source control method, an inspection method, and an exposure method.

Japanese Unexamined Patent Application Publication No. 2020-077007 describes a light source in which a target material is formed on the surface of a cylindrical member rotating around a rotation axis and the formed target material is irradiated with excitation light to emit illumination light.

Japanese Unexamined Patent Application Publication No. 2022-168463 describes a light source in which a target material, which is molten metal, is held with a centrifugal force on an inner wall of a crucible rotating around a rotation axis and the held target material is irradiated with excitation light to emit illumination light.

### SUMMARY

In a light source apparatus, relative positions of a light emitting point located near a target material surface and an optical member may fluctuate because of vibration caused when a target holding unit such as a cylindrical member or a crucible rotates, deformation of a structure itself, and consumption or supplement of the target material into the target holding unit. Accordingly, there may be a case in which light cannot be stably extracted from the light source apparatus.

An object of the present disclosure, which has been made in order to solve such a problem, is to provide a light source apparatus, an inspection apparatus, an exposure apparatus, a light source control method, an inspection method, and an exposure method that can improve stability of light extracted from the light source apparatus.

A light source apparatus according to the present disclosure includes: an input optical system including a first optical member configured to irradiate a target material with excitation light; an output optical system including a second optical member configured to extract light generated by irradiating the target material with the excitation light; a target holding unit configured to hold the target material; an acquiring unit configured to acquire a surface position of the target material; a driving unit configured to cause a position of a focusing point of at least one of the first optical member and the second optical member to vary; and a control unit configured to drive the driving unit based on the surface position.

In the light source apparatus, the acquiring unit may acquire the surface position as a relative position to the second optical member.

In the light source apparatus, the first optical member may irradiate the target material with the excitation light at an angle tilted from an axis perpendicular to a surface of the target material.

In the light source apparatus, the target holding unit may move the target material to an irradiation position irradiated with the excitation light according to the movement of the target holding unit.

In the light source apparatus, the acquiring unit may acquire the surface position in a peripheral position other than the irradiation position and predict the surface position in the irradiation position from the acquired surface position in the peripheral position to thereby acquire the surface position in the irradiation position.

In the light source apparatus, the target material may include molten metal, and the target holding unit may include a container, which houses the target material on an inside, and support the target material on an inner wall surface of the container with a centrifugal force.

In the light source apparatus, the acquiring unit may acquire the surface position of the target material based on a distance from a sensor to a surface of a liquid surface of the molten metal.

In the light source apparatus, the acquiring unit may predict the surface position in the irradiation position from the surface position in a position on a near side of the irradiation position with respect to a direction of the movement of the target holding unit, and the control unit may control the driving unit based on the predicted surface position.

In the light source apparatus, when the surface position varies at a predetermined period, the acquiring unit may predict the surface position based on the period, and the control unit may control the driving unit based on the predicted surface position.

In the light source apparatus, the first optical member may include a mirror configured to reflect the excitation light to the target material.

In the light source apparatus, the second optical member may include a collector mirror configured to reflect the generated light.

In the light source apparatus, the first optical member may include a mirror configured to reflect the excitation light to the target material, the second optical member may include a collector mirror configured to reflect the generated light, and the control unit may set a driving control amount for the first optical member larger than a driving control amount for the second optical member.

In the light source apparatus, the first optical member may include a condensing lens configured to condense the excitation light, and the control unit may control the driving unit to move the condensing lens in a direction parallel to an optical axis of the excitation light so that the position of the focusing point varies.

In the light source apparatus, the first optical member may include a mirror configured to reflect the excitation light to the target material and a condensing lens configured to condense the excitation light, when the surface position is a predetermined surface position, the control unit may control the driving unit to cause an optical axis of the mirror to vary so that the position of the focusing point to varies and control the driving unit to move the condensing lens in a direction parallel to an optical axis of the excitation light so that the position of the focusing point varies and, when the surface position is not the predetermined surface position, the control unit may control the driving unit to cause the optical axis of the mirror to vary so that the position of the focusing point varies and limit the movement of the condensing lens in the direction parallel to the optical axis of the excitation light.

In the light source apparatus, the light may include EUV light.

An inspection apparatus according to the present disclosure includes: the light source apparatus explained above; and an inspection optical system configured to inspect an inspection target with the light extracted from the output optical system.

In the inspection apparatus, the control unit may drive the driving unit such that the light scans a visual field region in the inspection target.

In the inspection apparatus, the control unit may drive the driving unit such that movement of the scan in a component of a short side direction of the visual field region having a rectangular shape in the inspection target stays within a fixed range.

An exposure apparatus according to the present disclosure includes: the light source apparatus explained above; and an exposure optical system configured to expose an exposure target with the light extracted from the output optical system.

In the exposure apparatus, the control unit may drive the driving unit such that the light scans an exposure region in the exposure target.

A light source control method according to the present disclosure includes: a step of holding a target material with a target holding unit; a step of acquiring a surface position of the target material with an acquiring unit; a step of a control unit allowing, based on the surface position, a driving unit to cause a position of a focusing point of at least one of a first optical member and a second optical member to vary; a step of causing an input optical system including the first optical member to irradiate the target material with excitation light; and a step of extracting, with an output optical system including the second optical member, light generated by irradiating the target material with the excitation light.

An inspection method according to the present disclosure includes a step of inspecting an inspection target with the light extracted by the light source control method explained above.

An exposure method according to the present disclosure includes a step of exposing an exposure target with the light extracted by the light source control method explained above.

According to the present disclosure, it is possible to provide a light source apparatus, an inspection apparatus, an exposure apparatus, a light source control method, an inspection method, and an exposure method that can improve stability of light extracted from the light source apparatus.

The above and other objects, features and advantages of the present disclosure will become more fully understood from the detailed description given hereinbelow and the accompanying drawings.

### BRIEF DESCRIPTION OF DRAWINGS

FIG. 1 is a sectional view illustrating a light source apparatus according to a first embodiment;

FIG. 2 is a perspective view illustrating a container functioning as a target holding unit in the light source apparatus according to the first embodiment;

FIG. 3 is a plan view illustrating the light source apparatus according to the first embodiment;

FIG. 4 is a diagram illustrating an acquiring unit, a driving unit, and a control unit in the light source apparatus according to the first embodiment;

FIG. 5 is a schematic diagram illustrating a relation between displacement of a surface position of a target material and movement of a bright spot in the light source apparatus according to the first embodiment and is a diagram viewed from a rotation axis direction of a container;

FIG. 6 is a schematic diagram illustrating a relation between the displacement of the surface position of the target material and the movement of the bright spot in the light source apparatus according to the first embodiment and is a diagram viewed from a direction orthogonal to the rotation axis;

FIG. 7 is a schematic diagram illustrating a visual field region detected by a detector of an inspection apparatus that detects generated light in the light source apparatus according to the first embodiment;

FIG. 8 is a schematic diagram illustrating cancellation of displacement of a surface position using a mirror in the light source apparatus according to the first embodiment and is a diagram viewed from the rotation axis direction of the container;

FIG. 9 is a schematic diagram illustrating the cancellation of the displacement of the surface position using the mirror in the light source apparatus according to the first embodiment and is a diagram viewed from the direction orthogonal to the rotation axis;

FIG. 10 is a schematic diagram illustrating a visual field region detected by the detector of the inspection apparatus that detects generated light in the light source apparatus according to the first embodiment;

FIG. 11 is a flowchart illustrating a light source control method using the light source apparatus according to the first embodiment; and

FIG. 12 is a configuration diagram illustrating an inspection apparatus including the light source apparatus according to the first embodiment.

### DESCRIPTION OF EMBODIMENTS

A specific configuration of an embodiment is explained below with reference to the drawings. The following explanation indicates a preferred embodiment of the present disclosure. The scope of the present disclosure is not limited to the embodiment explained below. In the following explanation, those with the same reference numerals and signs indicate substantially the same content.

### First Embodiment

A light source apparatus according to a first embodiment is explained. The light source apparatus in the present embodiment generates light such as illumination light and exposure light used for an optical apparatus such as an inspection apparatus and an exposure apparatus. The light source apparatus may be provided integrally with the optical apparatus or may be disposed near the optical apparatus as a separate body separated from the optical apparatus. When the optical apparatus is the inspection apparatus, the light source apparatus generates illumination light for illuminating an inspection target in the inspection apparatus. When the optical apparatus is the exposure apparatus, the light source apparatus generates exposure light for exposing an exposure target in the exposure apparatus.

The light source apparatus generates light such as illumination light and exposure light by irradiating a target material held by a target holding unit with excitation light. In the following explanation, as an example of the light source apparatus, a light source apparatus that sets molten metal held by a container as the target material is explained. Note that the light source apparatus is not limited to the light source apparatus that sets the molten metal held by the container as the target material and may be a light source apparatus that sets droplet-like liquid metal, solid material fixed to a cylindrical drum, or the like as the target material.

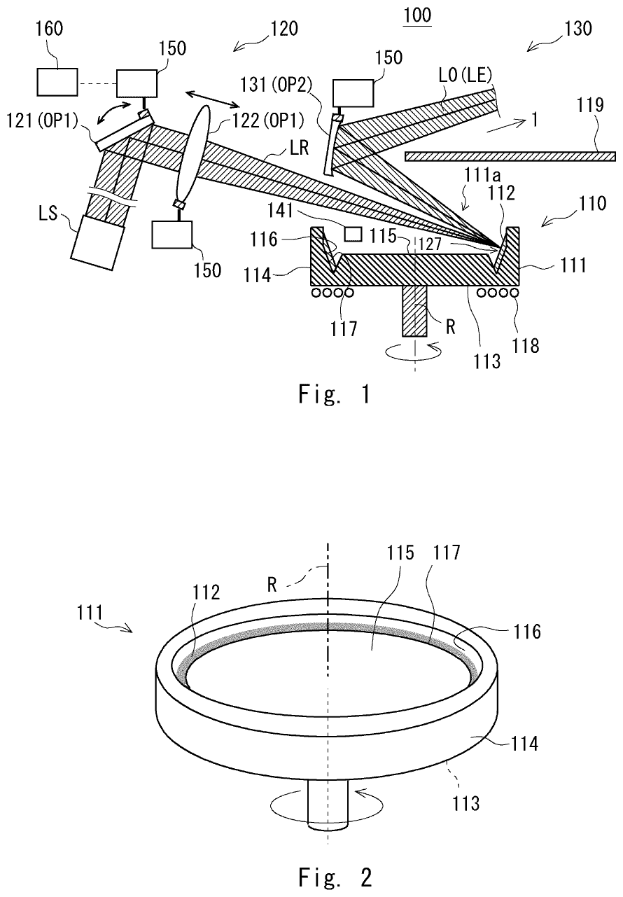

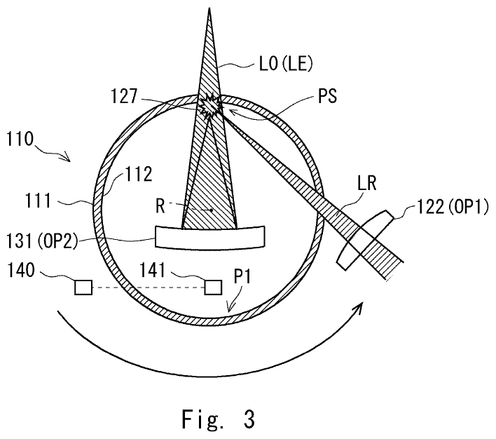

FIG. 1 is a sectional view illustrating a light source apparatus 100 according to the first embodiment. FIG. 2 is a perspective view illustrating a container 111 functioning as a target holding unit 110 in the light source apparatus 100 according to the first embodiment. FIG. 3 is a plan view illustrating the light source apparatus 100 according to the first embodiment. In FIG. 3, several members are omitted. As illustrated in FIGS. 1 to 3, the light source apparatus 100 includes a target holding unit 110, an input optical system 120, an output optical system 130, an acquiring unit 140, a sensor 141, a driving unit 150, and a control unit 160. In FIG. 1, the driving unit 150 is connected to a mirror 121, a condensing lens 122, and a collector mirror 131. However, the driving unit 150 does not always need to be connected to all of these optical members. The control unit 160 is connected to only several members to prevent the figure from becoming complicated. However, the control unit 160 may be connected to other members.

The target holding unit 110 holds a target material 112. The target holding unit 110 includes a container 111 such as a crucible. The container 111 can melt metal inside. The container 111 holds the target material 112 such as molten metal that generates plasma 127 with irradiation of excitation light LR. The excitation light LR is, for example, laser light including IR (Infrared) light.

Note that the target holding unit 110 is not limited to the container 111 and may be a cylindrical drum. In that case, the target holding unit 110 holds the target material 112 by fixing solid material, which becomes the target material 112, such as xenon (Xe) frozen on the surface of the drum.

The target material 112 may include molten metal. Note that the target material 112 is not limited to the molten metal held by the container 111 and may be solid metal, droplets, or the like if the target material 112 is a substance that generates the plasma 127 with irradiation of the excitation light LR. The molten metal is, for example, melted tin (Sn) or lithium (Li) but is not limited to tin and lithium if the molten metal generates the plasma 127 with irradiation of the excitation light LR.

The container 111 has a rotation axis R and rotates centering on the rotation axis R. The container 111 has, for example, a cylindrical shape with one opening closed. A closed portion of the container 111 is referred to as bottom 113. A cylindrical portion of the container 111 is referred to as cylindrical section 114. The surface on the inner side of the bottom 113 is referred to as bottom surface 115. The surface on the inner side of the cylindrical section 114 is referred to as inner wall surface 116. A groove 117 may be formed in a joining portion of the bottom 113 and the cylindrical section 114. Note that the container 111 may include a shape other than the above if the container 111 can hold the molten metal.

The target holding unit 110 supports the target material 112 on the inner wall surface 116 of the container 111 with a centrifugal force. The inner wall surface 116 formed to surround the rotation axis R may include a cylindrical portion having a constant distance to the rotation axis R or may include a cone-shaped (or mortar-shaped) portion further expanded to the outer side upward. For example, the cone-shaped portion of the inner wall surface 116 may be connected to the groove 117.

The light source apparatus 100 may include a heater 118 and a debris shield 119 besides the target holding unit 110. The target material 112 such as molten metal can be formed in the container 111 by heating of the heater 118. The debris shield 119 is disposed in an opening 111a of the container 111 to cover the target material 112.

The target material 112 also rotates around the rotation axis R according to the rotation of the container 111 around the rotation axis R. As illustrated in FIG. 3, for example, the target material 112 located, at time t1, in a position P1 facing the sensor 141 moves to an irradiation position PS at time t2 according to the rotation of the container 111. In this way, according to the movement (that is, the rotational movement) of the target holding unit 110, the target holding unit 110 moves the target material 112 to the irradiation position PS where the target material 112 is irradiated with the excitation light LR.

The input optical system 120 includes a first optical member OP1. The first optical member OP1 irradiates the target material 112 with the excitation light LR. The first optical member OP1 includes, for example, at least one of a mirror 121 and a condensing lens 122. Note that, if the first optical member OP1 is an optical member that irradiates the target material 112 with the excitation light LR, the first optical member OP1 is not limited to the mirror 121 and the condensing lens 122 and may also be any types of optical element including a laser LS that generates the excitation light LR.

The first optical member OP1 irradiates the target material 112 with the excitation light LR at an angle tilted from an axis perpendicular to the surface of the target material 112. Specifically, for example, the first optical member OP1 irradiates, at a tilted incident angle, the surface of the irradiation position PS with the excitation light LR. By irradiating the surface of the irradiation position PS with the excitation light LR at the tilted angle in this way, it is possible to suppress the influence of debris on the optical member including the collector mirror 131. A reason why the influence of debris on the optical member such as a collector mirror 131 can be suppressed is explained below.

When the surface of the target material 112 is irradiated with the excitation light LR from a direction orthogonal to the surface of the target material 112, the debris scatters in all directions centering on the direction orthogonal to the surface. Then, the debris is likely to adhere to the collector mirror 131 facing the irradiation position PS. On the other hand, when the surface of the irradiation position PS is irradiated with the excitation light LR at the tilted angle, the debris can be scattered in a reflection direction on the side opposite to an incident direction of the excitation light LR. Accordingly, it is possible to suppress the adhesion of the debris to the collector mirror 131. Further, the excitation light LR is irradiated at an incident angle tilted to the near side of the irradiation position PS with respect to the direction of the movement of the target holding unit 110. That is, the excitation light LR is irradiated from a direction having a component of an incident angle tilted to the near side in a surface orthogonal to the rotation axis R. Then, angular velocity in a rotating direction of the container 111 is applied in a direction in which the debris scatters. Therefore, it is possible to further scatter the debris in a reflection direction of the excitation light LR. In this way, it is possible to suppress the influence of the debris on the optical members such as the collector mirror 131.

The mirror 121 reflects, for example, the excitation light LR generated by the laser LS to the irradiation position PS of the target material 112. The mirror 121 may include a mirror such as a piezo steering mirror. Note that, the mirror 121 is not limited to the piezo steering mirror and may include a Galvano mirror, a polygon mirror, and the like if the mirror 121 can reflect the excitation light LR to the target material 112. The condensing lens 122 condenses the excitation light LR on the irradiation position PS of the target material 112.

The light source apparatus 100 may include the laser LS that generates the excitation light LR. On the other hand, the light source apparatus 100 may introduce, into the light source apparatus 100, the excitation light LR from the laser LS installed separately from the light source apparatus 100 on the outside of the light source apparatus 100. The excitation light LR is, for example, laser light including IR light. The excitation light LR may irradiate the target material 112 according to oscillation and stop of control of the control unit 160. For example, the excitation light LR is reflected by the mirror 121 and condensed by the condensing lens 122. Accordingly, the excitation light LR irradiates the target material 112.

The output optical system 130 includes a second optical member OP2. The second optical member OP2 extracts, from the light source apparatus 100, light L0 generated by irradiating the target material 112 with the excitation light LR. The second optical member OP2 includes, for example, the collector mirror 131. Note that, if the second optical member OP2 is an optical member that extracts the light L0 generated by irradiating the target material 112 with the excitation light LR, the second optical member OP2 is not limited to the collector mirror 131 and may also be any types of optical element including a second collector mirror (not illustrated) that further reflects the light L0 reflected by the collector mirror 131.

The collector mirror 131 reflects the light L0 generated from the target material 112 by the irradiation of the excitation light LR. The collector mirror 131 reflects, for example, EUV light LE generated by the irradiation of the excitation light LR. That is, the light L0 may include the EUV light LE. The EUV light LE is generated from the plasma 127 generated by irradiating the target material 112 with the excitation light LR. The EUV light LE generated from the plasma 127 generated by the target material 112 is emitted to an optical apparatus such as an inspection apparatus as illumination light. Thus, the illumination light includes the EUV light generated from the plasma 127.

The acquiring unit 140 acquires a surface position of the target material 112. The acquiring unit 140 is connected to the sensor 141 and acquires, from the sensor 141, a surface position of the target material 112 measured by the sensor 141. The acquiring unit 140 acquires a surface position of the target material 112 in the irradiation position PS where the excitation light LR irradiates the target material 112. The acquiring unit 140 may acquire a surface position measured by the sensor 141 in the irradiation position PS or, as explained below, may predict a surface position in the irradiation position PS from a surface position measured by the sensor 141 in a peripheral position. The acquiring unit 140 may predict the surface position of the target material 112 considering a tilt with respect to the rotation axis and vibration of the target holding unit 110.

The acquiring unit 140 may be a separate body separated from the sensor 141 or may be integrated with the sensor 141. Specifically, the sensor 141 may include, for example, a displacement meter, a high-speed camera, a low-speed camera, a quadripartite PD (Photo Diode), and a TDI (Time Delay Integration) camera. The acquiring unit 140 may combine other sensors with the sensor 141 such as the displacement meter to thereby acquire the surface position of the target material 112. Accordingly, the other sensors can supplement phase information that the sensor 141 such as the displacement meter cannot easily acquire.

The acquiring unit 140 may acquire the surface position of the target material 112 as a relative position to the second optical member OP2. Specifically, the acquiring unit 140 may acquire the surface position in the irradiation position PS of the target material 112 as the relative position to the second optical member OP2 or may acquire the surface position in the peripheral position as the relative position to the second optical member OP2. The acquiring unit 140 may acquire the surface position of the target material 112 based on the distance from the sensor 141 to the surface of a liquid surface of molten metal. The acquiring unit 140 may acquire the surface position of the target material 112 based on the thickness of the liquid surface of the molten metal from the inner wall surface 116. Note that, when the target material 112 is solid material fixed to a cylindrical drum, the acquiring unit 140 may acquire the surface position of the target material 112 based on a tilt and a vibration amount of the drum besides the thickness of the surface of the solid material from the upper surface (the uppermost surface) of the drum.

The acquiring unit 140 may acquire a surface position in a peripheral position other than the irradiation position PS. The peripheral position includes a portion other than the irradiation position PS on the inner wall surface 116 of the container 111. The acquiring unit 140 may predict the surface position of the irradiation position PS from the surface position in the peripheral position acquired from the sensor 141. Specifically, the acquiring unit 140 predicts the surface position in the irradiation position PS from a surface position in a position on the near side of the irradiation position PS with respect to the direction of the movement of the target holding unit 110. At this time, considering moving speed (rotating speed) of the target holding unit 110 and a distance between the irradiation position PS and the peripheral position, it is possible to predict the surface position in the irradiation position PS at a point in time (an irradiation point in time) when the excitation light reaches the irradiation position PS. The acquiring unit 140 predicts the surface position in the irradiation position PS in this way to thereby acquire the surface position in the irradiation position PS.

When the sensor 141 is disposed in a position facing the irradiation position PS to be able to measure and acquire the surface position in the irradiation position PS, the sensor 141 is likely to be affected by debris. Since the plasma 127 is generated in the irradiation position PS, the sensor 141 is likely to not accurately acquire the surface position. Therefore, the sensor 141 is disposed to face a peripheral position separated from the irradiation position PS. Accordingly, it is possible to suppress the influence of the debris and improve measurement accuracy of the surface position. For example, the sensor 141 may be disposed to face a position P1 on the side opposite to the irradiation position PS with respect to the rotation axis R. Note that the sensor 141 may be disposed to face a peripheral position other than the position P1 and to face the irradiation position PS if the influence of the debris can be reduced.

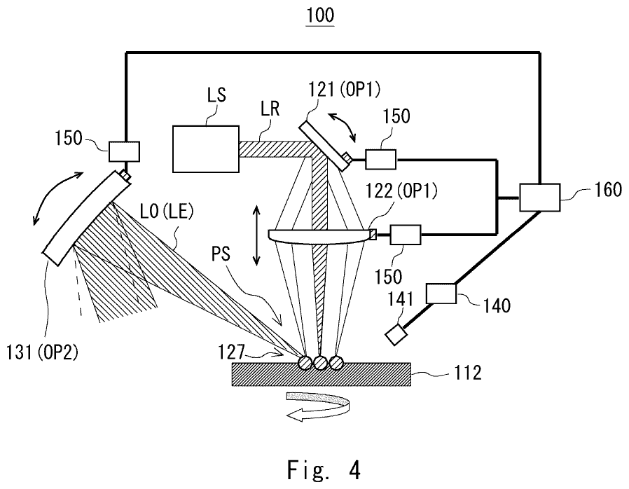

FIG. 4 is a diagram illustrating the acquiring unit 140, the driving unit 150, and the control unit 160 in the light source apparatus 100 according to the first embodiment. As explained above, the position of the sensor 141 is not limited to the position facing the irradiation position PS and may be a position facing the peripheral position such as the position P1. As illustrated in FIG. 4, the driving unit 150 causes the position of the focusing point of at least one of the first optical member OP1 and the second optical member OP2 to vary. The driving unit 150 is, for example, an actuator.

The driving unit 150 drives the first optical member OP1 to change an irradiation direction of the excitation light LR. For example, when the first optical member OP1 is the mirror 121, the driving unit 150 swings an angle of the mirror 121 with respect to the excitation light LR to perform beam scan. Specifically, the driving unit 150 changes a reflection surface of the mirror 121 such that the excitation light LR scans the surface of the target material 112 in a predetermined direction.

When the mirror 121 is a piezo steering mirror, the driving unit 150 may include a driving mechanism provided in the piezo steering mirror. When the mirror 121 is a Galvano mirror, a polygon mirror, and the like, the driving unit 150 may be a driving mechanism provided in the Galvano mirror, the polygon mirror, and the like. Note that, when there is another actuator having a short lead time and good controllability, the driving unit 150 may be the actuator.

The plasm 127 is generated in the irradiation position PS where the excitation light LR irradiates the target material 112. The generated plasma 127 is observed as a bright spot. The driving unit 150 causes the optical axis of the mirror 121 to vary to cause the position of the focusing point to vary. Accordingly, the driving unit 150 moves the bright spot at high speed to perform beam shaving. Thus, when the optical apparatus is an inspection apparatus, it is possible to improve uniformity and availability on a detector of the inspection apparatus. By causing the optical axis of the mirror 121 to vary, the driving unit 150 may cause the position of the focusing point to vary in at least two axial directions on the surface of the target material 112 in the irradiation position PS.

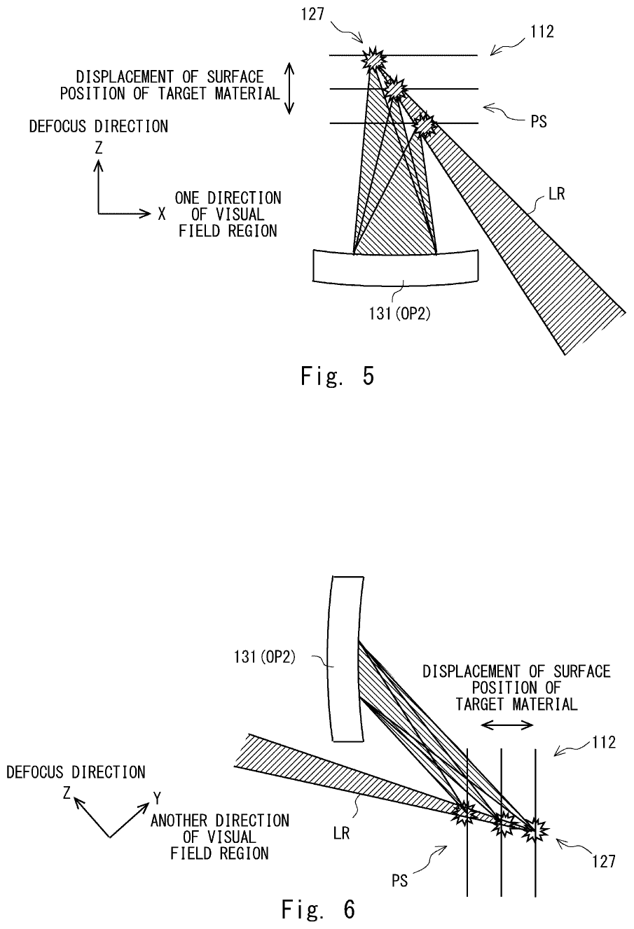

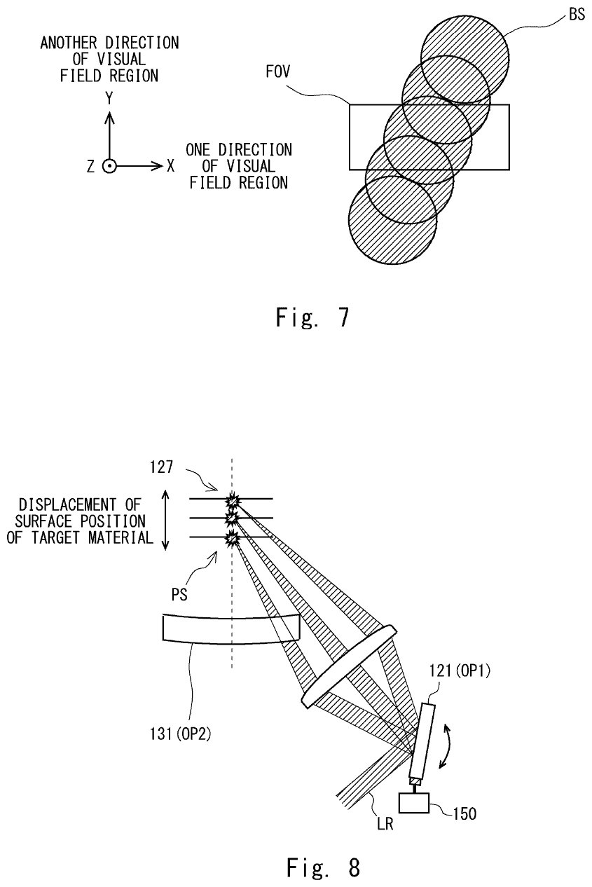

FIG. 5 is a schematic diagram illustrating a relation between displacement of the surface position of the target material 112 and the movement of the bright spot in the light source apparatus 100 according to the first embodiment and is a diagram viewed from the rotation axis R direction of the container 111. FIG. 6 is a schematic diagram illustrating the relation between the displacement of the surface position of the target material 112 and the movement of the bright spot in the light source apparatus 100 according to the first embodiment and is a diagram viewed from a direction orthogonal to the rotation axis R. FIG. 7 is a schematic diagram illustrating a visual field region FOV detected by the detector of the inspection apparatus that detects the generated light L0 in the light source apparatus 100 according to the first embodiment. Note that, here, displacement of a surface position of the target material 112 held by the container 111 is explained as an example. However, the same applies when a surface position of the target material 112 held by a cylindrical drum surface is conceived.

As illustrated in FIG. 7, for convenience of explanation, one direction in the visual field region FOV detected by the detector is represented as an X-axis direction and another direction orthogonal to the one direction is represented as a Y-axis direction. In that case, the Z-axis direction is a defocus direction of a beam spot BS. The defocus direction corresponds to the optical axis direction of the collector mirror 131. In FIGS. 5 and 6, a relation between a moving direction on the visual field region FOV of the beam spot BS of the light L0 generated from a bright spot of the plasma 127 and a moving direction of the plasma 127 generated in the target material 112 is illustrated.

The displacement of the surface position of the target material 112 includes surface vibration caused by vibration due to the rotation of the container 111 and the like propagating to the surface position and displacement due to consumption or supply of the target material 112. When the target material 112 is a solid such as frozen xenon, the displacement of the surface position of the target material 112 includes displacement due to the roughness and unevenness of the target surface.

As illustrated in FIGS. 5 to 7, when the excitation light LR is obliquely made incident on the surface position of the target material 112, if the surface position is displaced, the beam spot BS moves in directions having components of the X-axis direction and the Y-axis direction on the visual field region FOV. In this way, the displacement of the bright spot in the direction orthogonal to the optical axis of the collector mirror 131 is displacement in the X-axis direction and the Y-axis direction of the beam spot BS on the visual field region FOV.

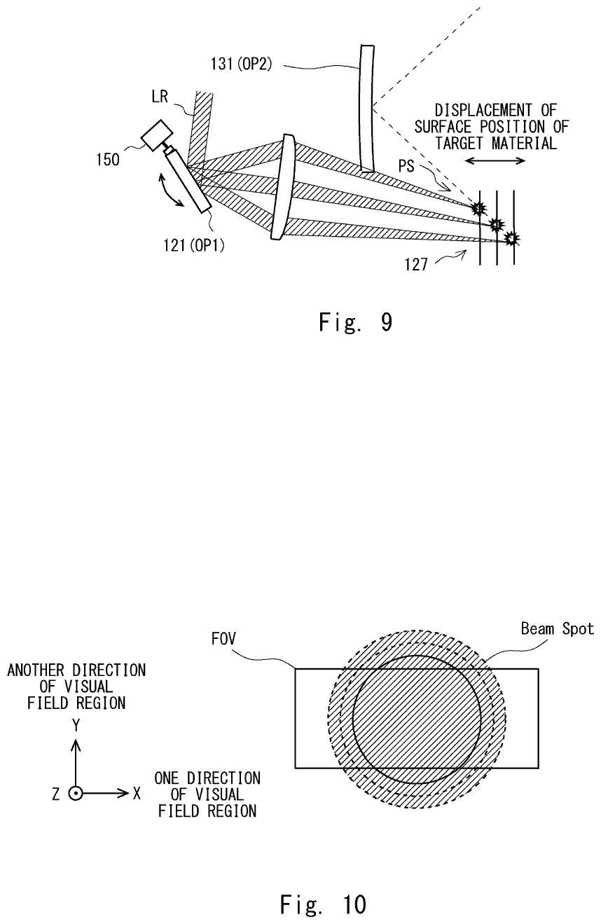

FIG. 8 is a schematic diagram illustrating cancellation of displacement of a surface position using the mirror 121 in the light source apparatus 100 according to the first embodiment and is a diagram viewed from the rotation axis R direction of the container 111. FIG. 9 is a schematic diagram illustrating the cancellation of the displacement of the surface position using the mirror 121 in the light source apparatus 100 according to the first embodiment and is a diagram viewed from a direction orthogonal to the rotation axis R. As illustrated in FIGS. 8 and 9, by causing the reflection surface of the mirror 121 such as a steering mirror to vary according to the displacement of the surface position, displacement of a component perpendicular to an optical axis of the mirror 121 can be cancelled. In the displacement of the surface position, surface vibration is displaced at a high frequency. Thus, the steering mirror that can be driven at a high frequency is suitable for cancellation of the surface vibration. A direction of displacement of the surface position due to consumption and supply of the target material 112 is the same as the direction of the surface vibration explained above. However, such displacement due to consumption and supply of the target material 112 is displacement at a low frequency. Thus, the driving unit 150 drives the first optical member OP1 at a low frequency.

FIG. 10 is a schematic diagram illustrating the visual field region FOV detected by the detector of the inspection apparatus that detects generated light in the light source apparatus 100 according to the first embodiment. As illustrated in FIG. 10, displacement of a bright spot in a direction parallel to an optical axis is defocus in the Z-axis direction of the beam spot BS. Therefore, the driving unit 150 moves the position of the condensing lens 122 in the direction parallel to the optical axis of the excitation light LR. Accordingly, the driving unit 150 causes the position of the focusing point of the excitation light LR to vary. As explained above, when the first optical member OP1 includes the condensing lens 122, the driving unit 150 moves the position of the condensing lens 122 in the direction parallel to the optical axis of the excitation light LR.

The driving unit 150 may drive the second optical member OP2 to change an extracting direction of the generated light L0 and/or change a focus position to the target holding unit 110. When the surface position of the target material 112 is displaced, an optical path of the light L0 generated from the target material 112 is displaced. Therefore, when the second optical member OP2 includes the collector mirror 131, the driving unit 150 swings an angle of the collector mirror 131 with respect to the bright spot of the light L0.

The control unit 160 drives the driving unit 150 based on the surface position of the target material 112 acquired by the acquiring unit 140. Here, the acquiring unit 140 acquires the surface position of the target material 112 as a relative position to the second optical member OP2. When it is predicted that the surface position acquired as the relative position to the second optical member OP2 will be displaced by a predetermined threshold or more, the control unit 160 controls the driving unit 150 such that the position of the focusing point of at least one of the first optical member OP1 and the second optical member OP2 varies. Therefore, even if the surface position (the thickness of the target material 112 from the inner wall surface 116 and the drum surface) is displaced, when the relative position is not displaced by the predetermined threshold or more, the control unit 160 may limit execution of the control of the driving unit 150.

The control unit 160 calculates a control amount based on the surface position acquired by the acquiring unit 140. Then, the control unit 160 drives the driving unit 150 based on the calculated control amount. The control unit 160 may include, for example, a control board. For example, the control unit 160 controls the driving unit 150 based on the calculated control amount such that the optical axis of the mirror 121 varies and the position of the focusing point varies. The control unit 160 controls the driving unit 150 based on the calculated control amount such that the condensing lens 122 is moved in the direction parallel to the optical axis of the excitation light LR and the position of the focusing point varies. Further, the control unit 160 controls the driving unit 150 based on the calculated control amount such that the optical axis of the collector mirror 131 varies and the optical axis of the generated light L0 varies. The control unit 160 may drive the driving unit 150 such that the light L0 scans the visual field region FOV in the inspection target of the inspection apparatus.

For example, the control unit 160 drives the driving unit 150 such that, in the movement of the scan in the component of the Y-axis direction illustrated in FIG. 7, for example, 80% of a track of the center of the beam spot BS stays within the visual field region FOV. A control amount serving as a target for the driving can be determined according to an experiment result or a simulation result in advance. Specifically, an experiment or a simulation is performed about to which degree the beam spot BS moves in the Y-axis direction according to which degree of displacement of the surface position and to which degree the beam spot BS moves in the Y-axis direction according to which degree of driving of the optical member. Note that 80% described above is an example. 80% of the beam sot BS itself may stay within the visual field region FOV. In this way, the control unit 160 drives the driving unit 150 such that movement of scan in a short side direction component of the visual field region FOV having a rectangular shape in the inspection target stays within a fixed range.

The control unit 160 may prioritize the control of the first optical member OP1 over the control of the second optical member OP2. For example, the control unit 160 may set a control amount of the first optical member OP1 larger than a control amount of the second optical member OP2. The control unit 160 may set the number of times of the control of the first optical member OP1 larger than the number of times of the control of the second optical member OP2. This is because the first optical member OP1 is more easily controlled than the second optical member OP2 and has less effects on the other optical members after being caused to vary by the driving unit 150.

The surface position of the target material 112 may suddenly increase or decrease. The surface position suddenly fluctuates, for example, when the target material 112 is supplied to the container and when the target material 112 is so called “return current” (the evaporated or scattered target is supplied to the container 111 again). In this case, the surface position increases or decreases to such an extent that it is even required to take defocus measures by the condensing lens 122 in addition to causing the mirror 121 to vary. Therefore, the surface position in the case of such sudden fluctuation is set as a predetermined surface position in advance. When the surface position of the target material 112 is the predetermined surface position, the control unit 160 controls the driving unit 150 to cause the optical axis of the mirror 121 so that the position of the focusing point varies and controls the driving unit 150 to move the condensing lens 122 in the direction parallel to the optical axis of the excitation light LR so that the position of the focusing point varies.

When the acquiring unit 140 predicts a surface position in the irradiation position PS from a surface position in the position P1 on the near side of the irradiation position PS with respect to the direction of the movement of the target holding unit 110, the control unit 160 calculates a control amount based on the predicted surface position. The control unit 160 feedforward-controls the driving unit 150 based on the calculated control amount.

The sensor 141 may measure a surface position in the irradiation position PS and the control unit 160 may control the driving unit 150 based on the measured surface position. However, it is conceivable that transmission of a control signal from the control unit 160 to the driving unit 150 or calculation of a control amount are not in time for control of the position of the focusing point of the optical member matching irradiation timing of the excitation light LR. Therefore, the control unit 160 controls (for example, feedforward-controls) the driving unit 150 based on the surface position in the irradiation position PS predicted from the surface position in the position P1. To cope with such an operation, the driving unit 150 may be designed to be capable of operating at several hundred hertz.

When the surface position varies at a predetermined period, the acquiring unit 140 may predict a surface position of the target material 112 based on the period and the control unit 160 may control (for example, feedforward-control) the driving unit 150 based on the predicted surface position. The control unit 160 may calculate a control amount based on the period and feedforward-control the driving unit 150. For example, when the fluctuation of the surface position includes surface vibration that vibrates at a predetermined period, the control unit 160 calculates a period of the surface vibration. The control unit 160 calculates a control amount based on the calculated period and feedforward-controls the driving unit 150.

### <Light Source Control Method>

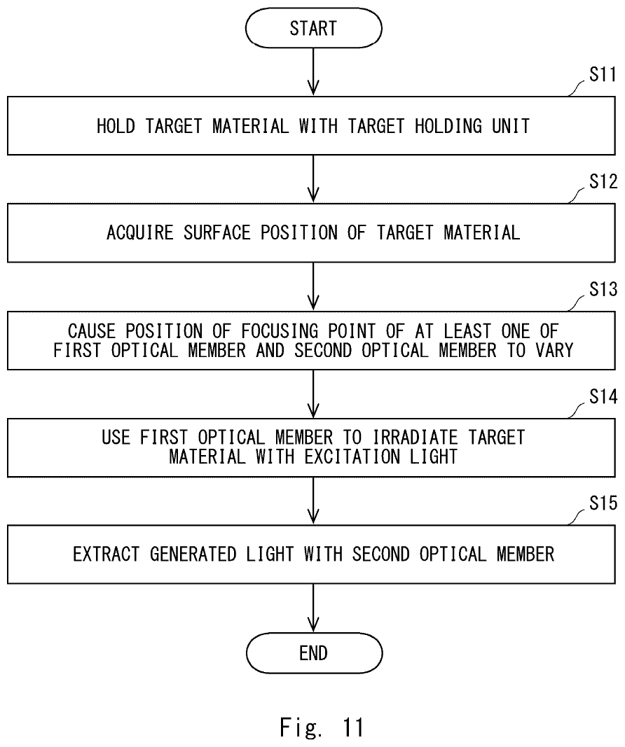

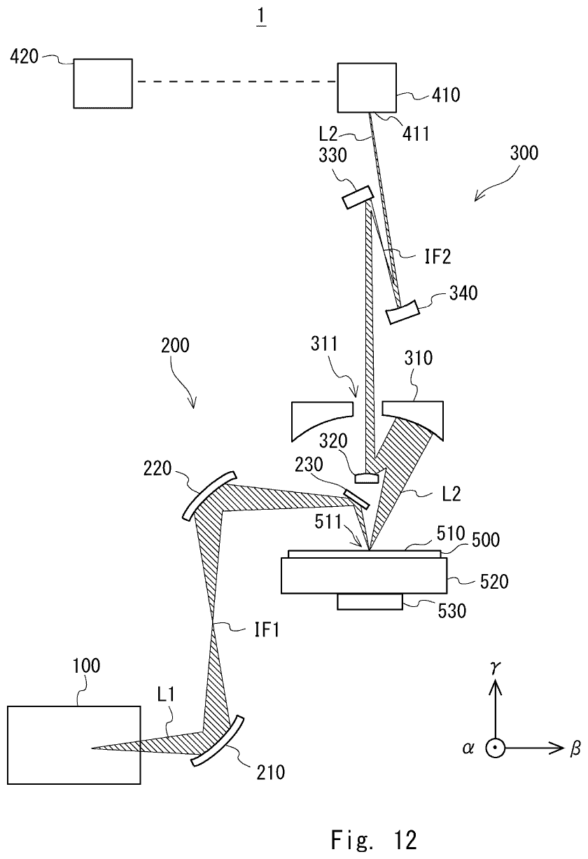

Subsequently, a light source control method using the light source apparatus 100 is explained. FIG. 11 is a flowchart illustrating a light source control method using the light source apparatus 100 according to the first embodiment. As illustrated in step S11 in FIG. 11, first, the light source apparatus 100 holds the target material 112 with the target holding unit 110. For example, the light source apparatus 100 holds, in the container 111 such as a melting pot, the target material 112 such as molten metal that generates the plasma 127 with irradiation of the excitation light LR.

Subsequently, as illustrated in step S12, the light source apparatus 100 acquires the surface position of the target material 112 with the acquiring unit 140. For example, the acquiring unit 140 may predict a surface position in the irradiation position PS from the surface position in the peripheral position measured by the sensor 141 to thereby acquire the surface position in the irradiation position PS.

Subsequently, as illustrated in step S13, the control unit 160 allows the driving unit 150 to cause the position of the focusing point of at least one of the first optical member OP1 and the second optical member OP2 to vary, based on the surface position acquired by the acquiring unit 140. The light source apparatus 100 causes the control unit 160 to calculate a control amount for driving the first optical member OP1 and (or) the second optical member OP2. Specifically, the light source apparatus 100 causes the control unit 160 to calculate, based on the surface position acquired by the acquiring unit 140, a control amount for the driving unit 150 to drive the first optical member OP1 and (or) the second optical member OP2. Then, the control unit 160 allows the driving unit 150 to cause the position of the focusing point of at least one of the first optical member OP1 and the second optical member OP2 to vary. For example, when the first optical member OP1 includes the mirror 121, the control unit 160 controls the driving unit 150 to cause the optical path of the mirror 121 to vary so that the position of the focusing point varies. When the first optical member OP1 includes the condensing lens 122, the control unit 160 controls the driving unit 150 to move the condensing lens 122 in the direction parallel to the optical axis of the excitation light LR so that the position of the focusing point varies. Further, when the second optical member OP2 includes the collector mirror 131, the control unit 160 controls the driving unit 150 based on the calculated control amount to cause the optical axis of the collector mirror 131 to vary such that the optical axis of the generated light L0 varies and/or to vary its focus point to the target holding unit 110.

Subsequently, as illustrated in step S14, the light source apparatus 100 uses the input optical system 120 including the first optical member OP1 to irradiate the target material 112 with the excitation light LR.

Subsequently, as illustrated in step S15, the light source apparatus 100 extracts, with the output optical system 130 including the second optical member OP2, the light L0 generated by irradiating the target material 112 with the excitation light LR. By controlling the light source apparatus 100 as explained above, it is possible to extract the light L0 used for the optical apparatus.

### <Optical Apparatus>

Subsequently, the optical apparatus is explained. The optical apparatus is explained using an inspection apparatus as an example of the optical apparatus.

FIG. 12 is a configuration diagram illustrating an inspection apparatus 1 including the light source apparatus 100 according to the first embodiment. As illustrated in FIG. 12, the inspection apparatus 1 includes an illumination optical system 200, an inspection optical system 300, a detector 410, and an image processing unit 420. Note that the inspection apparatus 1 may further include the light source apparatus 100. The inspection apparatus 1 is an apparatus that inspects a defect and the like of a sample 500 using, as the illumination light L1, the light L0 generated by the light source apparatus 100. The sample 500 is, for example, an EUV mask. Note that the sample 500 is not limited to the EUV mask and may be a semiconductor substrate or the like.

The illumination optical system 200 includes an ellipsoidal mirror 210, an ellipsoidal mirror 220, and a drop-in mirror 230. The inspection optical system 300 includes a concave mirror with hole 310, a convex mirror 320, a plane mirror 330, and a concave mirror 340. The concave mirror with hole 310 and the convex mirror 320 configure a Schwarzschild enlarging optical system.

The light source apparatus 100 generates illumination light L1. The illumination light L1 includes, for example, the EUV light LE having a wavelength of 13.5 nm that is the same as an exposure wavelength of the EUV mask to be the sample 500. Note that the illumination light L1 may include light other than the EUV light. The illumination light L1 generated from the light source apparatus 100 is reflected by the ellipsoidal mirror 210. The illumination light L1 reflected by the ellipsoidal mirror 210 travels while being narrowed and is condensed at a convergent point IF1. Thus, the ellipsoidal mirror 210 reflects, as convergent light, the illumination light L1 generated from the light source apparatus 100. The convergent point IF1 is a position conjugate with an upper surface 510 of the sample 500 such as the EUV mask and a detection surface 411 of the detector 410.

After passing the convergent point IF1, the illumination light L1 travels while expanding and is made incident on a reflection mirror such as the ellipsoidal mirror 220. Thus, the illumination light L1 reflected by the ellipsoidal mirror 210 is made incident on the ellipsoidal mirror 220 as divergent light via the convergent point IF1. The illumination light L1 made incident on the ellipsoidal mirror 220 is reflected by the ellipsoidal mirror 220, travels while being narrowed, and is made incident on the drop-in mirror 230. That is, the ellipsoidal mirror 220 reflects the incident illumination light L1 as convergent light. The ellipsoidal mirror 220 makes the illumination light L1 incident on the drop-in mirror 230. The drop-in mirror 230 is disposed right above the EUV mask. The illumination light L1 made incident on the drop-in mirror 230 and reflected is made incident on the sample 500. Thus, the drop-in mirror 230 makes the illumination light L1 incident on the sample 500 by reflecting, to the sample 500, the illumination light L1 reflected by the ellipsoidal mirror 220.

The ellipsoidal mirror 220 condenses the illumination light L1 on the sample 500. The illumination optical system 200 is installed to form an image of the bright spot of the light source apparatus 100 on the upper surface 510 of the sample 500 when the illumination light L1 illuminates the sample 500. Thus, the illumination optical system 200 is critical illumination. As explained above, the illumination optical system 200 illuminates the sample 500 such as the EUV mask using the critical illumination by the illumination light L1 generated by the light source apparatus 100.

The sample 500 is disposed on a stage 520. Here, a plane parallel to the upper surface 510 of the sample 500 is represented as αβ plane and a direction perpendicular to the αβ plane is represented as γ-axis direction. The illumination light L1 is made incident on the sample 500 from a direction tilting from the y-axis direction. That is, the illumination light L1 is made obliquely incident and illuminates the sample 500.

The stage 520 is a three-dimensional driving stage including a driving unit 530. The driving unit 530 can illuminate a desired region of the sample 500 by moving the stage 520 in the αβ plane. Further, the driving unit 530 can perform focus adjustment by moving the stage 520 in the γ-axis direction.

The illumination light L1 from the light source apparatus 100 illuminates an inspection region of the sample 500. The inspection region illuminated by the illumination light L1 is, for example, a 0.5 mm square. Note that the inspection region is not limited to the 0.5 mm square. The illumination light L1 is made incident on the sample 500 from a direction tilted with respect to the γ-axis direction. Light from the sample 500 illuminated by the illumination light L1 is made incident on the concave mirror with hole 310. In the following explanation, the light from the sample 500 illuminated by the illumination light L1 is explained as reflected light L2. Note that the light made incident on the concave mirror with hole 310 from the sample 500 is not limited to the reflected light L2 and may include diffracted light or the like. The reflected light L2 reflected by the sample 500 is made incident on the concave mirror with hole 310. A hole 311 is provided in the center of the concave mirror with hole 310. The concave mirror with hole 310 condenses the reflected light L2 from the sample 500 and reflects the condensed reflected light L2 as convergent light.

The reflected light L2 reflected by the concave mirror with hole 310 is made incident on the convex mirror 320. The convex mirror 320 reflects the reflected light L12 reflected by the concave mirror with hole 310 toward the hole 311 of the concave mirror with hole 310. The reflected light L2 having passed through the hole 311 is made incident on the plane mirror 330. The plane mirror 330 makes the reflected light L2 reflected by the convex mirror 320 incident as convergent light through the hole 311 of the concave mirror with hole 310. The reflected light L2 made incident on the plane mirror 330 is reflected by the plane mirror 330. The reflected light L2 reflected by the plane mirror 330 travels while being narrowed and is condensed at a convergent point IF2. Thus, the plane mirror 330 reflects the incident reflected light L2 as convergent light. The convergent point IF2 may be referred to as aperture stop. The convergent point IF2 is a position conjugate with the upper surface 510 of the sample 500 and the detection surface 411 of the detector 410.

After passing the convergent point IF2, the reflected light L2 travels while expanding and is made incident on the concave mirror 340. Thus, the reflected light L2 reflected by the plane mirror 330 as the convergent light is made incident on the concave mirror 340 via the focusing point IF2 as divergent light. The concave mirror 340 reflects the incident reflected light L2 to the detector 410 as convergent light. The reflected light L2 reflected by the concave mirror 340 is detected by the detector 410. As explained above, the inspection optical system 300 inspects the sample 500, which is the inspection target, with the light L1 extracted from the output optical system 130 of the light source apparatus 100. That is, the inspection optical system 300 condenses the reflected light L2 from the sample 500 illuminated by the illumination light L1 and guides the condensed reflected light L2 to the detector 410.

The detector 410 may include a TDI (Time Delay Integration) sensor. The detector 410 receives light from the sample 500 illuminated by the illumination light L1. A region on the sample 500 detected by the detector 410 is referred to as visual field region 511. The detector 410 receives the reflected light L2 from the visual field region 511 illuminated by the illumination light L1. The visual field region 511 may be included in the inspection region illuminated by the illumination light L1. The detector 410 acquires image data of the sample 500 such as the EUV mask. When the detector 410 includes a TDI sensor, the detector 410 includes a plurality of imaging elements linearly disposed side by side in one direction. The imaging elements are, for example, CCDs (Charge Coupled Devices). Note that the imaging elements are not limited to the CCDs.

The image data of the sample 500 acquired by the detector 410 is output to the image processing unit 420 and processed in the image processing unit 420. The image processing unit 420 may be, for example, a server apparatus or an information processing apparatus such as a personal computer.

The reflected light L2 includes information concerning a defect or the like of the sample 500. Regular reflected light of the illumination light L1 made incident on the sample 500 from a direction tilted with respect to the Z-axis direction is detected by the inspection optical system 300. When a defect is present in the sample 500, the defect is observed as a dark image. Such an observation method is referred to as bright field observation. Note that the inspection apparatus 1 may make the illumination light L1 incident on the sample 500 from the Z-axis direction and cause the inspection optical system 300 to detect the illumination light L1. When a defect is present in the sample 500, the defect is observed as a bright image. Such an observation method is referred to as dark field observation.

As explained above, the inspection apparatus 1 in the present embodiment includes the light source apparatus 100 explained above and the inspection optical system 300 that inspects an inspection target with the light L0 extracted from the output optical system 130. An inspection method using the inspection apparatus 1 includes a step of inspecting the inspection target with the light L0 extracted by the light source control method explained above. Note that the inspection apparatus 1 is explained as the optical apparatus. However, the optical apparatus may be an exposure apparatus. For example, the exposure apparatus includes the light source apparatus 100 explained above and an exposure optical system that exposes an exposure target with the light L0 extracted from the output optical system 130. The control unit 160 may drive the driving unit 150 such that the light L0 scans an exposure region in the exposure target. An exposure method using the exposure apparatus includes a step of exposing the exposure target with the light L0 extracted by the light source control method explained above.

Subsequently, effects of the present embodiment are explained. The light source apparatus 100 in the present embodiment drives the driving unit 150 with a control amount based on the surface position of the target material 112. Accordingly, the light source apparatus 100 causes the position of the focusing point of at least one of the first optical member OP1 and the second optical member OP2 to vary. Therefore, even if the surface position of the target material 112 fluctuates because of vibration and consumption of the target material 112, since the focusing point is caused to vary following the fluctuation, it is possible to improve stability of the light L0 extracted from the light source apparatus 100.

For example, originally, the first optical member OP1 such as the mirror 121 may be introduced to improve uniformity and availability on the detector 410 of the inspection apparatus 1. Specifically, the mirror 121 of the first optical member OP1 scans the illumination light L1 to uniformly illuminate the visual field region FOV. In the present embodiment, such a first optical member OP1 is also used to cancel the displacement of bright spot caused by the displacement of the surface position. Therefore, in the present embodiment, since it is possible to appropriately cope with fluctuation of the position of the bright spot due to the displacement of the surface position in the irradiation position PS caused by the fluctuation in the thickness of the target material 112, it is possible to realize stabilization of the intensity of the generated light L1 such as the EUV light LE.

The acquiring unit 140 acquires the surface position of the target material 112 as a relative position to the second optical member OP2. Therefore, it is possible to improve accuracy of the surface position. The acquiring unit 140 acquires a surface position in a peripheral position other than the irradiation position PS and predicts a surface position in the irradiation position PS from the acquired surface position in the peripheral position. Specifically, the acquiring unit 140 predicts a surface position in the irradiation position PS from a surface position in a position on the near side of the irradiation position PS with respect to the direction of the movement of the target holding unit 110. Thus, since the focusing point of the excitation light LR can be adjusted to the surface position in the irradiation position, it is possible to improve stability of the light L1. Further, the control unit 160 calculates a control amount based on the predicted surface position and feedforward-controls the driving unit 150. Accordingly, it is possible to cause the focusing point to vary following the displacement of the surface position.

The target holding unit 110 supports the target material 112 on the inner wall surface 116 with a centrifugal force of the rotating container 111. Thus, it is possible to uniformize the surface position of the target material 112 and improve stability of the light L1.

The control unit 160 may cause the optical axis of the mirror 121 to vary so that the position of the focusing point varies or may move the condensing lens 122 in the direction parallel to the optical axis of the excitation light LR so that the position of the focusing point varies. The control unit 160 may cause the optical axis of the collector mirror 131 to vary so that the optical axis of the generated light L0 varies and/or vary the position of the focusing point to the target holding unit 110. Since the control amount is calculated according to the fluctuation of the surface position in this way, it is possible to improve stability of the light L0.

Note, the acquiring unit 140 may acquire a reference position of the surface position of the target material 112. The reference position of the surface position of the target material 112 here may be a surface position of the target material 112 at a point in time when the target material 112 was irradiated with the excitation light LR last time or may be a surface position defined in advance when the optical apparatus was installed or activated. The reference position may be a position defined by another method. The control unit 160 drives the driving unit 150 based on the surface position of the target material 112 acquired by the acquiring unit 140. Here, the acquiring unit 140 may detect that the surface position of the target material 112 is displaced from the reference position. When it is detected that the displacement of the surface position from the reference position exceeds a predetermined threshold is acquired by the acquiring unit, the control unit 160 controls the driving unit 150 such that the position of the focusing point of at least one of the first optical member OP1 and the second optical member OP2 varies. For example, when it is detected that the displacement of the surface position from the reference position exceeds a predetermined threshold, the control unit controls the driving unit to cause an optical axis of the mirror to vary so that the position of the focusing point varies and control the driving unit to move the condensing lens in a direction parallel to an optical axis of the excitation light so that the position of the focusing point varies, and when it is not detected that the displacement of the surface position from the reference position exceeds a predetermined threshold, the control unit controls the driving unit to cause the optical axis of the mirror to vary so that the position of the focusing point varies and limits the movement of the condensing lens in the direction parallel to the optical axis of the excitation light.

The acquiring unit 140 may acquire the surface position of the target material 112 as a relative position to at least one of the first optical member OP1 and the second optical member OP2 (referred to as target optical member for convenience of explanation). Specifically, the acquiring unit 140 may acquire the surface position in the irradiation position PS of the target material 112 as the relative position to the target optical member or may acquire the surface position in the peripheral position as the relative position to the target optical member. The position sensor 141 may be attached to the target optical member or attached to the driving unit 150. Therefore, the position of the position sensor may change in conjunction with the driving of the target optical member.

The control unit 160 may drive the driving unit 150 based on the surface position of the target material 112 acquired by the acquiring unit 140. Here, the acquiring unit 140 acquires the surface position of the target material 112 as a relative position to the target optical member. When it is predicted that the surface position acquired as the relative position to the target optical member will be displaced by a predetermined threshold or more, the control unit 160 controls the driving unit 150 such that the position of the focusing point of at least one of the first optical member OP1 and the second optical member OP2 varies. Therefore, even if the surface position (the thickness of the target material 112 from the inner wall surface 116 and the drum surface) is displaced, when the relative position is not displaced by the predetermined threshold or more, the control unit 160 may limit execution of the control of the driving unit 150.

The embodiment of the present disclosure is explained above. However, the present disclosure includes appropriate modifications not spoiling the object and the advantages of the present disclosure and is not limited by the embodiment explained above. In the embodiment of the present disclosure, the target holding unit 110 is the container 111 such as the melting pot. However, the target holding unit 110 is not limited to this and may be a target holding unit that holds the target material 112 on the surface of a cylindrical object, a target holding unit that holds the target material 112 with a tape-like object, or the like. The components in the first embodiment may be combined as appropriate.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A light source apparatus comprising:
an input optical system including a first optical member configured to irradiate a target material with excitation light;
an output optical system including a second optical member configured to extract light generated by irradiating the target material with the excitation light;
a target holding unit configured to hold the target material;
an acquiring unit configured to acquire a surface position of the target material;
a driving unit configured to cause a position of a focusing point of at least one of the first optical member and the second optical member to vary; and
a control unit configured to drive the driving unit based on the surface position.

2. The light source apparatus according to claim 1, wherein the acquiring unit acquires the surface position as a relative position to the second optical member.

3. The light source apparatus according to claim 1, wherein the first optical member irradiates the target material with the excitation light at an angle tilted from an axis perpendicular to a surface of the target material.

4. The light source apparatus according to claim 1, wherein the target holding unit moves the target material to an irradiation position irradiated by the excitation light according to the movement of the target holding unit.

5. The light source apparatus according to claim 4, wherein
the acquiring unit acquires the surface position in a peripheral position other than the irradiation position and predicts the surface position in the irradiation position from the acquired surface position in the peripheral position to thereby acquire the surface position in the irradiation position.

6. The light source apparatus according to claim 4, wherein
the target material includes molten metal, and
the target holding unit includes a container, which houses the target material on an inside, and supports the target material on an inner wall surface of the container with a centrifugal force.

7. The light source apparatus according to claim 6, wherein the acquiring unit acquires the surface position of the target material based on a distance from a sensor to a surface of a liquid surface of the molten metal.

8. The light source apparatus according to claim 5, wherein
the acquiring unit predicts the surface position in the irradiation position from the surface position in a position on a near side of the irradiation position with respect to a direction of the movement of the target holding unit, and
the control unit controls the driving unit based on the predicted surface position.

9. The light source apparatus according to claim 1, wherein, when the surface position varies at a predetermined period, the acquiring unit predicts the surface position based on the period, and
the control unit controls the driving unit based on the predicted surface position.

10. The light source apparatus according to claim 1, wherein the first optical member includes a mirror configured to reflect the excitation light to the target material.

11. The light source apparatus according to claim 1, wherein the second optical member includes a collector mirror configured to reflect the generated light.

12. The light source apparatus according to claim 1, wherein
the first optical member includes a mirror configured to reflect the excitation light to the target material,
the second optical member includes a collector mirror configured to reflect the generated light, and
the control unit sets a driving control amount for the first optical member larger than a driving control amount for the second optical member.

13. The light source apparatus according to claim 1, wherein
the first optical member includes a condensing lens configured to condense the excitation light, and
the control unit controls the driving unit to move the condensing lens in a direction parallel to an optical axis of the excitation light so that the position of the focusing point varies.

14. The light source apparatus according to claim 1, wherein
the first optical member includes a mirror configured to reflect the excitation light to the target material and a condensing lens configured to condense the excitation light,
when the surface position is a predetermined surface position, the control unit controls the driving unit to cause an optical axis of the mirror to vary so that the position of the focusing point varies and control the driving unit to move the condensing lens in a direction parallel to an optical axis of the excitation light so that the position of the focusing point varies, and
when the surface position is not the predetermined surface position, the control unit controls the driving unit to cause the optical axis of the mirror to vary so that the position of the focusing point varies and limits the movement of the condensing lens in the direction parallel to the optical axis of the excitation light.

15. The light source apparatus according to claim 1, wherein the light includes EUV light.

16. An inspection apparatus comprising:
the light source apparatus according to claim 1; and
an inspection optical system configured to inspect an inspection target with the light extracted from the output optical system.

17. The inspection apparatus according to claim 16, wherein the control unit drives the driving unit such that the light scans a visual field region in the inspection target.

18. The inspection apparatus according to claim 16, wherein the control unit drives the driving unit such that movement of the scan in a component of a short side direction of the visual field region having a rectangular shape in the inspection target stays within a fixed range.

19. An exposure apparatus comprising:
the light source apparatus according to claim 1; and
an exposure optical system configured to expose an exposure target with the light extracted from the output optical system.

20. The exposure apparatus according to claim 19, wherein the control unit drives the driving unit such that the light scans an exposure region in the exposure target.

21. A light source control method comprising:
a step of holding a target material with a target holding unit;
a step of acquiring a surface position of the target material with an acquiring unit;
a step of a control unit allowing, based on the surface position, a driving unit to cause a position of a focusing point of at least one of a first optical member and a second optical member to vary;
a step of causing an input optical system including the first optical member to irradiate the target material with excitation light; and
a step of extracting, with an output optical system including the second optical member, light generated by irradiating the target material with the excitation light.

22. An inspection method comprising a step of inspecting an inspection target with the light extracted by the light source control method according to claim 21.

23. An exposure method comprising a step of exposing an exposure target with the light extracted by the light source control method according to claim 21.
